(19) United States
(12) Patent Application Publication
(10) Pub. No.: US 2009/0157191 A1
(43) Pub. Date: Jun. 18, 2009

(54) Resurfacing Femoral Head Component

(76) Inventors: Simon Collins, Tetbury (GB); Ross Geoffrey Vallis, New South Wales (AU)

Correspondence Address: Law Office of ROBERT C. KLINGER, 2591 Dallas Parkway, Suite 300, FRISCO, TX, 75034, US

(21) Appl. No.: 11/991,880

(22) PCT Filed: Nov. 17, 2006
(86) PCT No.: PCT/GB2006/004289

§ 371 Date: Mar. 11, 2008

(30) Foreign Application Priority Data
Nov. 18, 2005 (GB) 0523552.8

(51) Int. Cl. A61F 2/32

(52) U.S. Cl. 623/22.4

(57) ABSTRACT

A femoral component having a head portion with an outwardly facing bearing surface and an opposing inner surface configured to fit over a prepared femur The femoral component also includes a stem portion which is for insertion into the prepared femur, the stem portion having a first end for engagement with the inner surface of the head portion. The head portion is provided on the inner surface with engagement means suitable for releasable engagement with complementary engagement means provided at the first end of the stem portion. The stem portion comprises a non-resorbable portion comprising the engagement means and a resorbable portion. The engagement means may, for example, be screw threads, bayonet fittings or push or press fit fittings of complementary tapers.

The present invention relates to a femoral head component for use in Hip Resurfacing procedures.

Conventional Total Hip Replacement (THR) is a procedure for the treatment of arthritis of the hip, a condition which causes considerable pain and loss of movement. As is well known, the hip is a ball and socket joint which allows the upper leg to move from side to side, back to front, and to rotate. The joint is made up of the head of the femur (the ball) which fits into the acetabulum (the socket). In a healthy hip, both the head of the femur and the acetabulum are covered with cartilage which provides a smooth surface allowing the joint to move freely.

In conventional Total Hip Replacement the worn head is cut off the femur and replaced with a metal ball and stem in the shaft of the femur and a plastic cup is placed in the prepared acetabulum. Both elements are typically anchored to the bone by “bone cement”. This has become a very common surgical procedure with some 45,000 hip replacements being carried out in the UK every year.

The plastic used to form the cup is inert and so is well tolerated by the body. Nevertheless, as the metal ball rubs against the plastic cup, tiny particles of the plastic are worn away. This plastic debris causes an irritation. Furthermore, as the particles get between the bone and the artificial joint, this irritation causes surrounding bone to be absorbed by the body, leading to a loosening of the artificial joint. In older people, with a lower activity level, this may not happen for twenty or more years, but in younger, more active patients, this may happen much sooner.

To overcome these problems in younger, more active patients requiring hip replacement, a different type of implant was needed. In 1991 a procedure for metal-on-metal (MoM) resurfacing of the hip was proposed. This has two major differences from a conventional THR. The first is that both components are typically made from metal, preferably Cobalt Chrome. By eliminating the plastic cup of a conventional THR, and making both parts of the bearing surface of metal, the resurfaced hip is expected to last much longer and therefore to be more suitable for higher demanding patients. The second difference is that the procedure is very bone conserving, since the head of the femur is simply reshaped and “resurfaced”, rather than removed. However, should the device need replacing at some time in the future, this has previously required the use of a conventional THR.

Since many patients who benefit from hip resurfacing are young it would however be preferable if the surgeon had other options available to him instead of a THR. In particular, it would be preferable if the surgeon could also have the option of using a further resurfacing femoral component, so that ultimate revision to a THR is delayed, hence conserving bone and thereby buying further time for the patient. Therefore, it is desired to provide an improved femoral component for use in the hip resurfacing procedure.

### SUMMARY OF THE INVENTION

According to a first aspect of the present invention there is provided a resurfacing femoral component comprising; a head portion having an outwardly facing bearing surface and an opposing inner surface configured to fit over a prepared femur; and a stem portion for insertion into the prepared femur, the stem portion having a first end for engagement with the inner surface of the head portion, the head portion being provided on the inner surface with engagement means for releasable engagement with complimentary engagement means provided at the upper end of the stem portion.

Advantageously the stem portion is made of a resorbable material.

It is also envisaged that the stem portion may be made of a mesh like material to encourage bone ingrowth.

According to a second aspect of the present invention there is provided a resurfacing femoral component comprising; a head portion having an outwardly facing bearing surface and an opposing inner surface configured to fit over a prepared femur; and a stem portion projecting from said inner surface for insertion into the prepared femur, at least part of said stem portion being made of resorbable material.

Advantageously, the stem portion comprises a non-resorbable core surrounded by resorbable material.

In an alternative embodiment the stem portion is wholly made of resorbable material.

The stem portion may be attached to the head portion by bonding, with adhesive or may be moulded thereto.

Advantageously, the head portion is provided on the inner surface with engagement means for releasable engagement with complimentary engagement means provided on a first end of the stem portion.

The engagement means previously described may comprise a threaded projection adapted to be received within a correspondingly threaded recess, the threaded projection extending from one of the inner surface of the head portion or the first end of the stem portion and the threaded recess being provided in the other.

Alternatively, the engagement means may comprise a frustoconical projection adapted to be received within a correspondingly shaped recess, the frustoconical projection extending from one of the inner surface of the head portion or the first end of the stem portion and the recess being provided in the other.

In a further alternative embodiment the engagement means is a bayonet type fitting.

In a yet further alternative, the engagement means comprise a projection adapted to be received in a correspondingly shaped recess with either a push fit or press fit engagement, the projection extending from one of the inner head surface of the head portion or the first end of the stem portion and the recess being provided in the other.

Advantageously, the stem portion comprises a non-resorbable portion comprising said engagement means and a resorbable portion.

In one embodiment, the non-resorbable portion comprises a first portion including the complimentary engagement means, the resorbable portion extending from an under surface of the first portion. The resorbable portion may be attached to the underside of the first portion by bonding, adhesive or moulding therewith, or the underside may be provided with engagement means for releasable engagement with complimentary engagement means provided on the resorbable portion.

In an alternative, the non-resorbable portion comprises a core which extends longitudinally of the stem portion and is surrounded by the resorbable portion. The core may be made of a mesh-like material to encourage bone ingrowth. Any of the head portion, core or first portion may be made of metal or ceramic.

Advantageously, the complimentary engagement means provided at the upper end of the stem further comprise a central guide member. In one embodiment the guide member is a female guide hole. In an alternative embodiment the guide member is a male guide projection.

Preferably the resorbable material is biocompatible.

More preferably the resorbable material is polylactic material or polyglycolic acid.

Advantageously, the inner surface of the head portion is coated with an osteoconductive coating.

Advantageously, the non-resorbable part of the stem portion, which in use is located within the femur, may be coated with an osteoconductive coating.

According to a third aspect of the present invention there is provided a method of revising a two-part resurfacing femoral component, the method comprising the steps of: disengaging the engagement means; removing the head portion; and attaching a head portion.

Advantageously the method further comprises the step of preparing the femoral head between the removal of the head portion and the attachment of the head portion.

In one embodiment the method further comprises treating the head portion after it has been removed and then reattaching the treated head portion.

In an alternative embodiment the head portion which is attached is different to the head portion which is removed.

According to a fourth aspect of the invention there is provided a method of inserting a two-part resurfacing femoral component the method comprising: preparing the femur to receive a resurfacing femoral component; providing a two-part resurfacing femoral component; inserting the stem portion into the femur and attaching the head portion to the inserted stem portion.

### BRIEF DESCRIPTION OF THE DRAWINGS

Preferred embodiments of the invention will now be described, by way of example, with reference to the accompanying drawings, in which:

FIG. 1 shows a side view of a resurfacing femoral head component of the prior art;

FIG. 2 shows a cross-section of the femoral head component of FIG. 1 along a longitudinal plane.

FIG. 3 shows a cross-section along a longitudinal plane of a femoral head component according to an embodiment of the present invention showing engagement means which are provided between a separable head portion and stem portion;

FIG. 4 shows an enlarged view of the engagement means of FIG. 3 according to one embodiment of the invention;

FIG. 5 shows an enlarged view of the engagement means of FIG. 3 according to an alternative embodiment of the present invention;

FIG. 6 shows a cross-section along a longitudinal plane of a femoral head component according to a further embodiment of the present invention showing engagement means which are provided between a separable head portion and stem portion;

FIG. 7a shows a cross-section through a longitudinal plane of a femoral head component according to a yet further embodiment of the present invention, the femoral head component comprising engagement means between a separable head portion and stem portion;

FIG. 7b shows an enlarged view of the engagement means of FIG. 7a according to one embodiment of the present invention;

FIG. 7c shows an enlarged view of an alternative version of engagement means for use in a femoral head component;

FIG. 8 shows a cross-section through a longitudinal plane of a femoral head component according to a still further embodiment of the present invention; and

FIG. 9 shows a cross-section along a longitudinal plane of yet another embodiment of femoral head component according to the present invention.

### DETAILED DESCRIPTION OF THE DRAWINGS

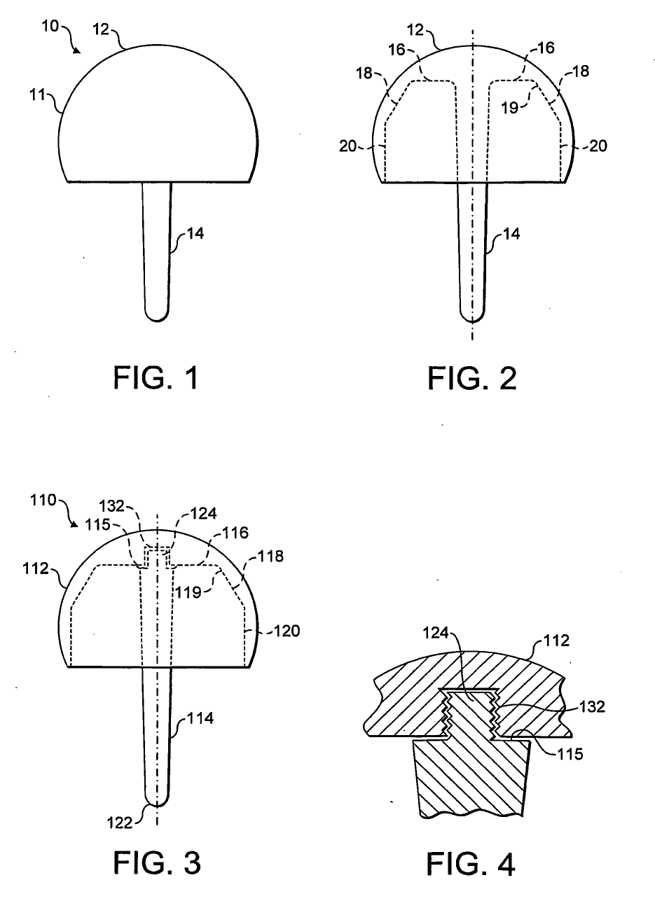

A conventional resurfacing femoral component 10 is shown in FIGS. 1 and 2 and comprises a head portion 12 having a bearing surface 11, which is a substantially spherical portion (generally slightly greater than a hemisphere), and a stem 14. In use the stem 14 is positioned within the femur and is used to ensure that the femoral head is correctly positioned whilst giving post operative stability in the first few months after implantation to the implant. As shown in FIG. 2, at its upper end the stem 14 merges with a radially outwardly extending top skirt surface 16 which in turn merges with a radially outwardly and downwardly depending upper skirt surface 18, which in turn merges with a downwardly depending lower skirt surface 20. Together the top skirt surface 16, the upper skirt surface 18 and the lower skirt surface 20 form a skirt like inner surface 19 which fits over the prepared head of the femur. At its lower end the lower skirt surface 20 merges with the bearing surface 11. Together the bearing surface 11, lower skirt surface 20, upper skirt surface 18 and top skirt surface 16 form the head portion 12. The resurfacing femoral component 10 is chosen by the surgeon to suit each individual patient based on the most suitable diameter of the bearing surface.

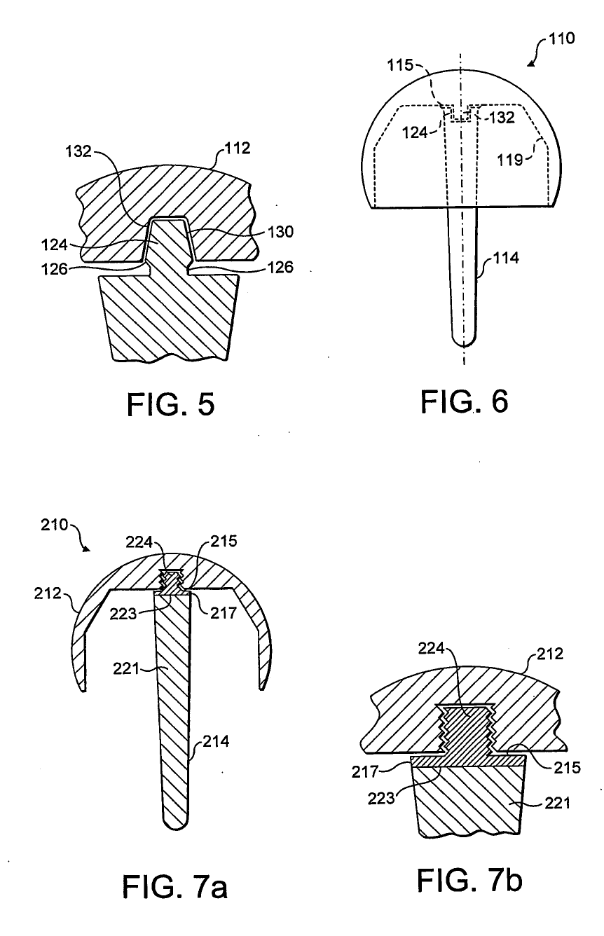

FIG. 3-5 show an embodiment of the present invention for an improved resurfacing femoral component 110 which comprises a separable stem portion 114 and head portion 112. FIGS. 4 and 5 show examples of the way in which the head portion 112 and stem portion 114 may be attached.

As shown in FIG. 3, the stem portion 114 takes the form of a solid substantially cone like structure with a preferably rounded tip 122. At the end of the stem portion 114 remote from the tip 122 is an upper surface 115 from which extends an attachment member 124. One example of a suitable attachment member 124 is shown in FIG. 4 to be a threaded spigot extending from the upper surface 115. An alternative attachment member 124 is shown in FIG. 5, where the attachment member 124 comprises a cylindrical portion 126 which extends from the upper surface 115 and merges with a radially outwardly extending chamfer 128 before in turn merging with a frustoconical portion 130 which tapers in a direction away from the upper surface 115.

As previously described, the head portion 112, comprises a bearing surface 111 which merges at its lower edge with an upwardly extending lower skirt surface 120 which in turn merges with a radially inwardly and upwardly extending upper skirt surface 118 which in turn merges with a radially inwardly extending top skirt surface 116 to form an inner surface 119. In the present invention the top skirt surface 116 includes an attachment portion 132 which extends into the head portion 112 to form a bore.

The attachment portion 132 may take the form of a threaded bore 132 for engagement with the threaded attachment member 124, as shown in FIG. 4. Alternatively, the attachment portion 132 may be a frustoconical shaped bore 132 for engagement, preferably by a push or press fit, with the frustoconical attachment member 124, as shown in FIG. 5. It will also be understood that other attachment means may be used to releasably attach the head portion 112 and stem portion 114 of a two part femoral head component 110, such as a bayonet type fitting, a press or push fit connector or other suitable attachment means.

Alternatively, as shown in FIG. 6, the attachment portion 132 may take the form of a projection extending downwardly into the stem portion 114 from the upper surface 115. The projection being threaded, or having a frustoconical shape or taking other forms such as a bayonet fitting as described above. In this arrangement, the top skirt portion 116, instead of merging with an upwardly extending attachment portion 124, will include a centrally positioned recess which is either threaded or frustoconically shaped to engage with the projection provided on the head portion 112 as appropriate.

The provision of a two part resurfacing femoral component 110 as described above provides the advantage that should the bearing surface 111 become worn or be damaged so that further surgery becomes necessary, the surgeon will have the option of simply removing and replacing the head portion 112. This is possible since the attachment member 124 and attachment portion 132, can be disengaged. Once the head portion 112 is removed the surgeon then re-prepares the femoral head or neck, for example by bone cuts or using a suitable head cutter and head chamfer cutter, and attaches a new head portion 112. This is done by engaging the attachment portion 132 provided on the head portion 112 with the attachment member 124 provided on the stem portion 114.

In an alternative embodiment of the present invention, the stem portion 114 may be wholly or partially made of a resorbable material. The resorbable material is preferably biocompatible and may, for example, be polylactic or polyglycolic acid. The advantage of a resorbable material is that following implantation, the resorbable part of the stem portion 114 is resorbed over, say, a period of 3 months to 1 year when the stem is required for initial stability and is then replaced by bone. This permits a more natural loading of the head of the femur and provides good bone stock should a revision operation become necessary.

When the stem portion 114 is resorbable it may either be attached to the bearing surface portion 112 using mechanical attachment means as previously described or, alternatively, may be attached by other means, such as adhesive, which is preferably biocompatible.

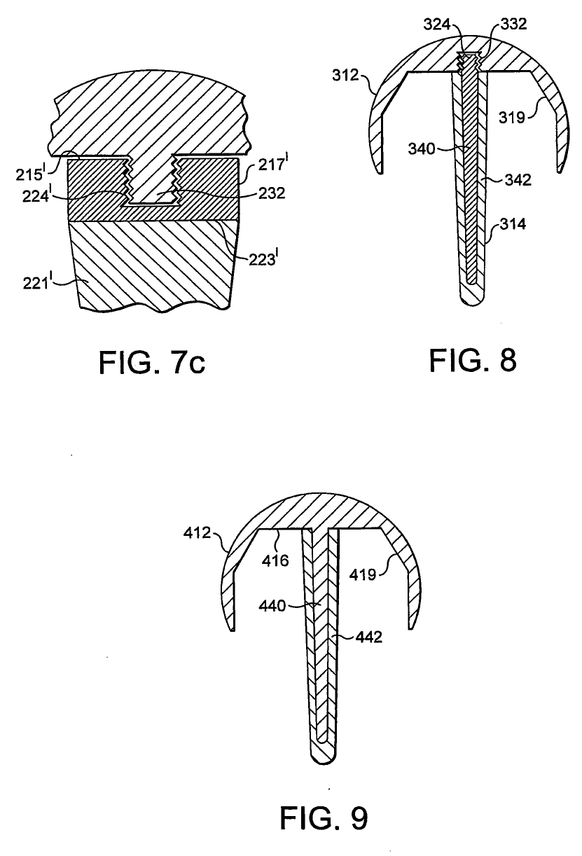

As mentioned previously the stem portion 114 may be either fully resorbable or may be comprised partly of a resorbable material. Examples of mixed material stems which are particularly advantageous are shown in FIGS. 7 and 8.

In FIGS. 7a and 7b the stem portion 214 comprises a non-resorbable, e.g. metallic, attachment member 224 extending from an upper surface 215 of a plate 217. A resorbable lower stem portion 221 is attached to an under surface 223 of the plate 217, for example by bonding, with adhesive or via injection moulding. Alternatively mechanical releasable attachment means such as a complementary threaded recess and projection, bayonet fitting may be provided on the under surface 223 of the plate and an upper surface of the resorbable lower stem portion 221.

Thus, when the stem portion 214 is implanted the lower stem portion 221 will be resorbed and replaced by new bone growth, but the plate 217, and the attachment member 224 extending therefrom, will remain in position and can be used to attach a replacement head portion 212 if necessary. As shown in FIG. 7c it is also possible that a plate 217′ may comprise an attachment member 224′ depending from upper surface 215′ in the form of a recess into which a projecting attachment portion 232′ can be inserted. As with the embodiment shown in FIG. 7b, a lower stem portion 221′ depends from an under surface 223 of the plate 217′ which is of a resorbable material. The lower stem portion 221′ may again be attached either by adhesive or other suitable non-releasable attachment means or by a complementary threaded recess and projection or other suitable releasable attachment means. Again, after implantation the lower stem portion 221′ will be resorbed and replaced with bone, whilst the plate 217′ will become embedded into the bone.

Of course it will be understood that the plate 217 or 217′ need not extend across the whole upper surface of the stem but may be circumferentially surrounded by resorbable material.

It will also be understood that the attachment means may also take the form of a frustoconical attachment portion and attachment member or other suitable forms as previously discussed. Furthermore, the attachment member may also be formed of resorbable material rather than a separate plate arrangement if desired.

In a further embodiment, shown in FIG. 8, a stem portion 314 comprises a non-resorbable core 340, for example made of metal. The core 340 is significantly smaller in diameter than a conventional resurfacing stem portion so that the stress shielding caused by the core 340 when in situ will be less than that caused by a conventional stem. The core comprises a male or female attachment member 324 as has been discussed previously. The stem portion 314 further comprises a resorbable portion 342 which surrounds the core 340. The resorbable portion 342 is attached to the core 340, for example by bonding, with adhesive or by injection moulding. As the resorbable portion 342 is absorbed the bone will grow around the core 340 and maintain the core 340 in position. This means that should a revision operation be necessary the attachment member 324 is in position. Therefore, once the worn or damaged head portion 312 is removed and the femoral head or neck re-prepared as discussed previously, a new head portion 312 can be attached to the existing attachment member.

In a further alternative of the present invention, as shown in FIG. 9, the head portion 412 may further comprise a core 440 of a non-resorbable material which depends from a top skirt surface 416 of an inner surface 419 of the head portion 412. The core 440 and head portion 412 being a one-piece component. The core 440 is then surrounded by a resorbable portion 442, which is attached to the core 440 by any suitable method, for example adhesive or bonding.

Preferably the core 340, 440 as shown and described in relation to FIGS. 8 and 9 is of an open mesh form to promote ingrowth of bone as the resorbable portion of the stem 414, 314 is absorbed.

Where the femoral head component is of a two-part construction, as discussed in particular in relation to FIGS. 3-8, the attachment means provided on the stem portion preferably further comprises a central guide hole. The surgeon may then insert a Guide Rod into this guide hole when re-preparing the femoral head to attach a new head portion.

In any of the described embodiments all or part of the inner surface 119, 219, 319, 419 may be provided with an osteoconductive coating, such as hydroxyapite, for use in a cementless implant procedure. Furthermore, any non-resorbable part of the stem, which in use will be located in the femur, may also be provided with an osteoconductive coating to encourage fixation. Alternatively, it will be understood that the implant may be fixed using a suitable bone cement.

Any of the previously discussed two-part resurfacing femoral components 110, 210, 310 may be implanted by firstly inserting the stem portion 114, 214, 314 into the prepared femur and then attaching the head portion 112, 212, 312 thereto.

If the surgeon is performing a revision operation and removes the head portion 112, 212, 312, as discussed previously, it is also envisaged that, rather than placing a new head portion 112, 212, 312 on to the stem, the removed head portion 112, 212, 312 may be treated. For example any debris may be removed or the bearing surface 111 may be repolished. The treated head portion 112, 212, 312 is then reattached to the corresponding attachment means extending from, or into, the femur.

1. A resurfacing femoral component comprising;
a head portion having an outwardly facing bearing surface and an opposing inner surface configured to fit over a prepared femur; and
a stem portion for insertion into said prepared femur, the stem portion having a first end for engagement with the inner surface of the head portion,
the head portion being provided on the inner surface with engagement means for releasable engagement with complimentary engagement means provided at the first end of the stem portion, the stem portion comprising a non-resorbable portion comprising said engagement means and a resorbable portion.

2. The resurfacing femoral component of claim 1, wherein the non-resorbable portion of the stem portion is made of a mesh like material to encourage bone ingrowth.

3. The resurfacing femoral component of claim 1, wherein said engagement means comprise a threaded projection adapted to be received within a correspondingly threaded recess, the threaded projection extending from one of the inner surface of the head portion or the first end of the stem portion and the threaded recess being provided in the other.

4. The resurfacing femoral component of claim 1, wherein said engagement means comprise a frustoconical projection adapted to be received within a correspondingly shaped recess, the frustoconical projection extending from one of the inner surface of the head portion or the first end of the stem portion and the recess being provided in the other.

5. The resurfacing femoral component of claim 1, wherein said engagement means is a bayonet type fitting.

6. The resurfacing femoral component of claim 1, wherein said engagement means comprise a projection adapted to be received in a correspondingly shaped recess with either a push fit or press fit engagement, the projection extending from one of the inner head surface of the head portion or the first end of the stem portion and the recess being provided in the other.

7. The resurfacing femoral component of claim 1, wherein said non-resorbable portion comprises a first portion including said complimentary engagement means, said resorbable portion extending from an under surface of said first portion.

8. The resurfacing femoral component of claim 7, wherein said first portion further comprises engagement means provided on the under surface for releasable engagement with complimentary engagement means provided on said resorbable portion.

9. The resurfacing femoral component of claim 7, wherein said resorbable portion is attached to said under surface by bonding, with adhesive or is moulded thereto.

10. The resurfacing femoral component of claim 1, wherein said non-resorbable portion comprises a core which extends longitudinally of the stem portion and is surrounded by said resorbable portion.

11. The resurfacing femoral component of claim 10, wherein said core is made of a mesh-like material to encourage bone ingrowth.

12. The resurfacing femoral component of claim 1, wherein said non-resorbable portion comprises a plate extending across at least a portion of the first end.

13. The resurfacing femoral component of claim 1, wherein any of the head portion, core or first portion are made of metal or ceramic.

14. The resurfacing femoral component of claim 1, wherein the complimentary engagement means provided at the upper end of the stem further comprise a central guide member.

15. The resurfacing femoral component of claim 14, wherein the guide member is a female guide hole.

16. The resurfacing femoral component of claim 14, wherein the guide member is a male guide projection.

17. The resurfacing femoral component of claim 1, wherein the resorbable material is biocompatible.

18. The resurfacing femoral component of claim 17, wherein the resorbable material is polylactic material or polyglycolic acid.

19. The resurfacing femoral component of claim 1, wherein the inner surface of the head portion is coated with an osteoconductive coating.

20. A resurfacing femoral component of claim 1, wherein the non-resorbable part of the stem portion which in use is located within the femur is coated with an osteoconductive coating.

21. A method of revising a two-part resurfacing femoral component according to any preceding claim, the method comprising:
disengaging the engagement means;
removing the head portion; and
attaching a head portion.

22. The method of claim 21, further comprising re-preparing the femoral head between the removal of the head portion and the attachment of the head portion.

23. The method of claim 21 further comprising;
treating the head portion after it has been removed; and
reattaching the treated head portion.

24. The method of claim, wherein the head portion which is attached is different to the head portion which is removed.

25. A method of inserting a two-part resurfacing femoral components the method comprising:
preparing the femur to receive a resurfacing femoral component;
providing a two-part resurfacing femoral component according to claim 1;
inserting the stem portion into the femur; and
attaching the head portion to the inserted stem portion.
